(19) United States
(12) Patent Application Publication
(10) Pub. No.: US 2022/0273601 A1
(43) Pub. Date: Sep. 1, 2022

(54) COLLAGEN PRODUCTION PROMOTER, WRINKLE IMPROVER, TOPICAL SKIN CARE COMPOSITION, AND BEAUTY FOOD OR BEVERAGE

(71) Applicant: REFINE HOLDINGS CO., LTD., Gifu (JP)

(72) Inventors: Makoto TSUBOI, Tokyo (JP); Kunimitsu KAYA, Tokyo (JP); Fujio SHIRAISHI, Tokyo (JP); Seiji SAKAMOTO, Tokyo (JP); Izumi IWATA, Tokyo (JP)

(73) Assignee: REFINE HOLDINGS CO., LTD., Gifu (JP)

(21) Appl. No.: 17/631,125

(22) PCT Filed: Jul. 30, 2020
(86) PCT No.: PCT/JP2020/029254

§ 371 Date: Jan. 28, 2022

(30) Foreign Application Priority Data
Jul. 31, 2019 (JP) 2019-140770

(51) Int. Cl. A61K 31/20; A61K 9/00; A61Q 19/08; A61K 8/36; A23L 33/12

(52) U.S. Cl. CPC A61K 31/20; A61K 9/0014; A61Q 19/08; A61K 8/361; A23L 33/12

(57) ABSTRACT

Provided is a collagen production promoter comprising, as an active ingredient, a triglyceride represented by the following formula (I), wherein R¹, R² and R³ each denotes a saturated fatty acid residue, at least one of which is a pentadecanoic acid residue. The triglyceride comprising the pentadecanoic acid promotes the production of skin fibroblasts and collagen, and can be used as a topical skin care composition and a beauty food or beverage for improving the condition of skin.

### CROSS REFERENCE

This application claims priority based on Japanese Patent Application No. 2019-140770 filed in Japan on Jul. 31, 2019, the entire contents of which are incorporated herein by reference in their entirety. In addition, all the contents described in all patents, patent applications, and documents cited in the present application are incorporated herein by reference in their entirety.

### TECHNICAL FIELD

The present invention relates to a collagen production promoter, a wrinkle improver, a topical skin care composition, and a beauty food or beverage. More specifically, the present invention relates to a collagen production promoter, a wrinkle improver, and topical skin care compositions and beauty foods or beverages for promoting collagen production and/or improving wrinkle of skin, which each comprises a specific triglyceride compound as an active ingredient.

### BACKGROUND ART

The epidermis and dermis of the skin are composed of epidermal cells, fibroblasts, and extracellular matrix such as collagen that supports the skin structure from the outside of these cells. In young skin, the proliferation of fibroblasts is active, and the interaction between skin tissues maintains homeostasis, which ensures water retention, flexibility, and elasticity, keeping the skin taut, shiny, and fresh in appearance. However, under the influence of certain external factors such as exposure to ultraviolet lights, significant dryness of the air, excessive skin cleansing, and the like, or with advancing age, the proliferative capacity of fibroblasts decreases, causing a decrease in the production of collagen, a major component of the extracellular matrix, as well as a decrease in elasticity due to cross-linking. As a result, the skin's moisturizing function and elasticity decline, and abnormal exfoliation of keratin begins, causing the skin to lose its tone and luster and exhibit skin aging symptoms such as roughness and wrinkle formation. Thus, the changes associated with skin aging, such as wrinkle formation, dullness, loss of texture, and loss of elasticity, are thought to be deeply related to the decreased proliferative capacity of fibroblasts. Based on this idea, various proposals have been made for synthetic and natural cosmetics and quasi-drugs that can promote the proliferation of fibroblasts and prevent or improve skin aging.

For example, a collagen production stimulator containing N-benzoylglycylglycine as an active ingredient (see Patent literature 1), a topical skin care product containing a silicic acid derivative (see Patent literature 2), and an topical skin care product containing phenylpropanoid glycosides (see Patent literature 3) have been reported.

On the other hand, there is a known method by using algae of the genus Aurantiochytrium of producing a mixture of triglycerides containing odd-chain fatty acids as major components and triglycerides containing highly unsaturated fatty acids (see, for example, Patent literature 4). Most of the fatty acids in the bodies of mammals and birds are fatty acids with an even number of carbons, and odd-chain fatty acids are present in only 0.1 to a few percent of the body fat, muscles, organs, milk, and eggs of humans and domestic animals and poultry. Odd-chain fatty acids, like even-chain fatty acids, can be used as an energy source through (3-oxidation, but they also function to maintain the TCA cycle normally by converting propionyl-CoA, the last product of odd-chain fatty acids in the (3-oxidation system, to succinyl-CoA, which is replenished to the TCA cycle. It is expected to reduce muscle pain caused by muscle damage during and after exercise (see Non-patent literature 1) and to improve mitochondrial energy metabolism, thereby improving metabolic diseases and aging (see Non-patent literature 2).

There are also reports of therapeutic methods for Alzheimer's disease (AD) and aging by using a ketone producing diet containing odd-chain triglycerides to improve cognitive function, increase circulating ketone bodies, and decrease amyloid-β (Aβ) deposition in AD patients (see Patent literature 5). However, due to the relative difficulty in obtaining triglycerides containing odd-chain fatty acids, they were not yet known to be effective in the cosmetic field, especially in improving collagen production and skin wrinkles.

### CITATION LIST

### Non-Patent Literature

[Non-patent literature 1] Shimomura Y. et al., Effects of squat exercise and branched-chain amino acid supplementation on plasma free amino acid concentrations in young women, J. Nutr. Sci. Vitaminol. (2009) 55, 288-291.
[Non-patent literature 2] Pfeuffer M. and Jaudszus A. (2016) Pentadecanoic and Heptadecanoic Acids: Multifaceted Odd-Chain Fatty Acids, Adv Nutr. (2016) 15; 7(4): 730-4.

### PATENT LITERATURE

[Patent literature 1] JP2014-55116 A
[Patent literature 2] JP2015-221768 A
[Patent literature 3] JP2016-44160 A
[Patent literature 4] JP2016-89025 A
[Patent literature 5] JP2013-516416 A

### SUMMARY OF INVENTION

### Technical Problem

Wrinkles are one of the biggest cosmetic concerns for people of all ages. Wrinkles are caused by a decrease in the production of type I collagen in the skin, resulting in a decrease in the amount of type I collagen. In other words, it is known that wrinkles can be improved or prevented if the production of type I collagen can be promoted in human fibroblasts. One of the objects of the present invention is to find a new substance that can contribute such a collagen production-promoting effect, and to provide a wrinkle improver using the new substance, as well as a topical skin care composition and beauty food or beverage for promoting collagen production and/or improving wrinkles.

### Solution to Problem

As a result of intensive research and investigation to solve the above problems, we, the applicants discovered that certain naturally occurring triglyceride compounds affect skin fibroblasts and collagen production, and came to the conclusion that the use of these triglyceride compounds can provide novel topical skin compositions and beauty foods and beverages that improve the condition of the skin. In more detail, we discovered that the triglyceride compound acts on human fibroblasts to cause them to produce type I collagen significantly. We found that by using this triglyceride compound, which is highly effective in promoting collagen production and improving wrinkles, as an active ingredient, topical skin compositions and beauty foods and beverages that are effective in preventing and improving skin aging can be obtained, leading to the completion of the present invention.

That is, in one embodiment, the present invention provides a collagen production promoter comprising, as an active ingredient, a triglyceride represented by the following formula (I):

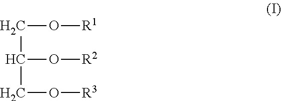

wherein R¹, R² and R³ each denotes a saturated fatty acid residue, at least one of which is a pentadecanoic acid residue. For the triglyceride of formula (I), it is preferable that R¹ and R² or R¹ and R³ are pentadecanoic acid residues.

In the above embodiment, any one of R¹, R² and R³ may be a myristic acid residue (C14), a palmitic acid residue (C16) or a margaric acid residue (C17). The collagen production promoter in a preferred embodiment is a mixture comprising a triglyceride of formula (I) in which all of R¹, R² and R³ are pentadecanoic acid residues, and a triglyceride of formula (I) in which any two of R¹, R² and R³ are pentadecanoic acid residues, and another is a myristic acid residue or a palmitic acid residue.

In another embodiment of the present invention, a wrinkle improver comprising a triglyceride represented by the above formula (I) as an active ingredient is provided. In still another embodiment, a topical skin care composition comprising a triglyceride represented by the above formula (I) as an active ingredient is shown. This topical skin care composition is preferably used for promoting collagen production and/or improving wrinkles.

In another aspect of the present invention, a beauty food or beverage comprising a triglyceride represented by the above formula (I) as an active ingredient is provided. This beauty food or beverage is preferably used for promoting collagen production and/or improving wrinkles.

In yet another aspect of the present invention, there is provided a method for promoting production of skin collagen in a subject, which comprises administering to the subject an effective amount of triglyceride represented by the above formula (I).

### Advantageous Effects of Invention

The triglyceride represented by the above formula (I) according to the present invention, hereinafter, the triglyceride compounds represented by formula (I) may be collectively referred to as “pentadecanoic acid triglyceride” or “PdATG” for simplicity, may be used as a collagen production promoter and a wrinkle improver, and is useful as topical skin care products and beauty foods and beverages for this purpose.

### BRIEF DESCRIPTION OF DRAWINGS

FIG. 1 shows the results of examining the effect of pentadecanoic acid triglyceride on the production of type I collagen in human fibroblasts.

FIG. 2A shows the measurement result of transepidermal water loss (TEWL) as a result of evaluating the moisturizing effect of oral administration of pentadecanoic acid triglyceride using hairless mice.

FIG. 2B shows the measurement result of stratum corneum water content as a result of evaluating the moisturizing effect of oral administration of pentadecanoic acid triglyceride using hairless mice.

### DESCRIPTION OF EMBODIMENTS

Hereinafter, the pentadecanoic acid triglyceride, which is the active ingredient of the present invention, will be described first, and then the production method and various uses thereof will be described.

### (Active Ingredient)

In this specification, “pentadecanoic acid triglyceride” means an ester of at least one pentadecanoic acid and a glycerol, and includes a triglyceride wherein at least one, preferably any two, of R¹, R² and R³ shown in Formula (I) below, e.g., R¹ and R² or R¹ and R³, and even more preferably R¹, R² and R³, are pentadecanoic acid residues. The binding position(s) of pentadecanoic acid(s) to the glyceride may be any of the positions 1 to 3.

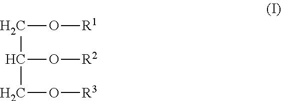

In the formula, if at least one of the saturated fatty acid residues represented by R¹, R² and R³ is a pentadecanoic acid residue, the other two may independently be saturated fatty acid residues other than pentadecanoic acid residues. “Saturated fatty acid” is a general term for fatty acids that do not have double or triple bonds within the molecule and is represented by the chemical formula CₙH₂ₙ₊₁COOH. Although not particularly limited, in the chemical formula of this saturated fatty acid, n is, for example, an integer of 4 to 26, preferably an integer of 11 to 19, and more preferably an integer of 14 or 16. When one of saturated fatty acid residues represented by R¹, R² and R³ is a pentadecanoic acid residue and the other two are saturated fatty acid residues other than pentadecanoic acid residue, these saturated fatty acids may be the same or different from each other. These saturated fatty acids can be straight-chain or branched saturated fatty acids. For example, linear saturated fatty acids, such as butyric acid (C4), valeric acid (C5), caproic acid (C6), enanthic acid (C7), caprylic acid (C8), pelargonic acid (C9), capric acid (C10), lauric acid (C12), myristic acid (C14), pentadecanoic acid (C15), palmitic acid (C16), margaric acid (C17), stearic acid (C18), arachidic acid (C20), behenic acid (C22), lignoceric acid (C24), and cerotic acid (C26), and branched saturated fatty acids such as 2-hexyl decanoic acid (C16), 13-methylpentadecanoic acid (C16), and 16-methylheptadecanoic acid (C18) are enumerated.

The pentadecanoic acid triglycerides in one preferred embodiment include both of a triglyceride of formula (I) above, where all of R¹, R² and R³ are pentadecanoic acid residues, and a triglyceride of formula (I) above, where any two of R¹, R² and R³ are pentadecanoic acid residues and the other one is a myristic acid residue or a palmitic acid residue. The content ratio of both in this mixture is not particularly limited, but the mass ratio is preferably 1:2 to 2:1 and more preferably approximately 1:1. In addition, each of these is contained in 10% or more by mass, preferably 20% or more by mass, relative to the total amount of triglycerides. Furthermore, it is more preferred that the mixture of triglycerides containing two or more residues of pentadecanoic acid contains more than 50% by mass of the oil or fat.

It is more preferable that the mixture of triglycerides containing two or more residues of pentadecanoic acid contains 50% by mass or more in the fat and oil, but even if the content of triglycerides containing two or more residues of pentadecanoic acid is 50% by mass or less, the objective can be achieved by increasing the amount of intake. Therefore, the active ingredient of the present invention may exist in the form of a mixture of triglycerides containing two or more residues of pentadecanoic acid, and is at least 1% by mass or more, preferably 50% by mass or more, and more preferably 90% by mass or more in purity relative to the total amount of triglycerides, thereby the mixture itself can exhibit its function as an active ingredient.

The active ingredient of the present invention may exist in the form of a mixture together with triglycerides other than the compound represented by the formula (I), and is at least 1% by mass, preferably 50% by mass or more, more preferably 90% by mass or more, based on the total amount of triglycerides, thereby the mixture itself can exert its function as an active ingredient.

### (Action and Effect)

While the pentadecanoic acid triglyceride, the active ingredient of the present invention, contains at least one pentadecanoic acid in the molecule, an odd-chain fatty acid such as pentadecanoic acid has a function of maintaining the TCA cycle normally. Fatty acids are oxidized in the body and become two-carbon (C2) acetyl-CoA, which enters the citric acid cycle (TCA cycle), reducing the cofactors NAD (nicotinamide adenine dinucleotide) and FAD (flavin adenine dinucleotide) to NADH₂ and FADH₂, and producing ATP by the electron transfer system. Here, in the case of fatty acids having an even number of carbon atoms, all carbon chains are decomposed into C2 acetyl-CoA and used in the TCA cycle, but in the case of odd-chain fatty acids such as pentadecanoic acid, C3 propionyl-CoA remains at the end of the degradation.

Propionyl-CoA is converted to C4, methyl-malonyl-CoA, which is then converted to succinyl-CoA, a member of the TCA cycle, by the enzyme methylmalonyl-CoA mutase with vitamin B12 as a cofactor. The succinyl-CoA introduced into the TCA cycle is converted to succinate by the enzyme succinyl-CoA synthase. This reaction produces a signal transducer, GTP (guanosine triphosphate). In the TCA cycle, this is the only reaction that produces GTP. ATP is not produced in the TCA cycle.

GTP binds to membrane proteins called G-proteins to transduce signals, and GTP-bound active G-proteins play an important role in activating various physiological functions of cells. Therefore, it is suggested that odd-chain fatty acids are involved in ATP production starting from C2 acetyl-CoA as well as in the activation of cellular physiological functions starting from C3 propionyl-CoA.

Since the molecules that make up the TCA cycle are also implicated in various other metabolic pathways, the TCA cycle may not function adequately due to a lack of one of its components. In such a case, the TCA cycle is replenished with succinyl-CoA produced by the degradation of odd-chain fatty acids as described above (this phenomenon is called anaplerosis). Conversely, if there is an excess of molecules in the TCA cycle, a cataplerotic reaction occurs, in which one of the constituent molecules is removed, and the TCA cycle is maintained normally. Odd-chain fatty acids function to maintain the TCA cycle normally by replenishing succinyl-CoA via propionyl-CoA.

In addition, according to the non-patent literature 2, it has been suggested that very long chain fatty acids (VLCFAs) synthesized from odd-chain fatty acids such as pentadecanoic acid are substrates for sphingoglycolipids in the brain and other tissues. Sphingolipids have various physiological functions such as skin barrier formation, neurological function, glucose tolerance, bacterial toxin/virus recognition, immunity, angiogenesis, and osteogenesis, and have been reported to be associated with various pathological conditions such as skin diseases, neurological diseases, metabolic syndrome, and cancer.

The active ingredient of the present invention has at least one, and preferably two or more of these odd-chain fatty acids, especially pentadecanoic acid, in its molecule, which is thought to exert the collagen production-promoting and wrinkle-improving effects described below when applied to the skin. In general, free fatty acids are toxic to cells, but the active ingredient of the present invention exists in the form of triglyceride, which is highly stable, and has a sustained release effect of pentadecanoic acid in vivo and is not toxic even when administered in large amounts, making it suitable as an topical skin care composition, and a beauty food or beverage.

### (Method for Producing Pentadecanoic Acid Triglyceride)

The pentadecanoic acid triglyceride, which is the active ingredient of the present invention, may be chemically synthesized or naturally occurring. Sources of naturally occurring pentadecanoic acid triglycerides include lipids produced in the body by living organisms, such as livestock and poultry fats, fish and shellfish oils, vegetable oils, or lipid-producing microorganisms. The genus Aurantiochytrium of Labyrinthula algae is particularly preferred because they are heterotrophic algae that live in brackish water and have the characteristic of assimilating nutrients in water to produce lipids and accumulate them in their cells.

For algae of the genus Aurantiochytrium, it is preferable to use strains with an excellent ability to produce the desired triglycerides. Such algal strains may be naturally collected and isolated, cloned through mutation induction and screening, or established using genetic recombination technology. Although not particularly limited, for example, the Aurantiochytrium strain mh1959 used in the example described below has the property of accumulating, in its cells, a large amount of triglycerides containing pentadecanoic acid (PDA), which is an odd-chain fatty acid, and those containing docosahexaenoic acid (DHA) and docosapentaenoic acid (DPA), which are highly unsaturated fatty acids. This strain is a desirable microorganism for the production of pentadecanoic acid triglyceride.

The cultivation of the above Aurantiochytrium algae is carried out using methods established in the art. That is, ordinary maintenance culture is carried out according to established methods by seeding the algae into a medium with appropriately prepared components. The medium for cultivating Aurantiochytrium essentially contains salts, carbon sources, and nitrogen sources. In general, the so-called GTY medium (10-40 g/L of artificial seawater, 20-100 g/L of D(+)glucose, 10-60 g/L of tryptone, and 5-40 g/L of yeast extract) is used for culturing microalgae.

Carbon sources include sugars such as glucose, fructose, and sucrose. These carbon sources are added, for example, at a concentration of 20-120 g per liter of the medium.

The algae of the genus Aurantiochytrium is a marine algae, and an appropriate amount of artificial seawater is added to the culture medium. Preferably, the artificial seawater is added so that the final salinity of the culture medium is about 10% (v/v) to about 100% (v/v) of seawater (salinity 3.4% (w/v)), e.g., salinity is about 1.0% to 3.0% (w/v).

In general, various nitrogen sources can be added to the culture medium of microalgae, such as organic nitrogen such as monosodium glutamate and urea, or inorganic nitrogen such as ammonium acetate, ammonium sulfate, ammonium chloride, sodium nitrate, ammonium nitrate, etc., or biogenic digests such as yeast extracts, cornstarch liquor, polypeptone, peptone, and tryptone. In particular, cell extracts obtained by extracting liquid components from various animal cells are preferred as nitrogen sources to be added to the medium used for culturing algae of the genus Aurantiochytrium. The use of cell extracts, which are rich in nutrients such as cell-derived amino acids, nucleic acids, vitamins, and minerals, and are available at low cost, is extremely advantageous when cells must be mass cultured on an industrial scale to obtain cultured cell products.

However, when using a medium prepared on the basis of cell extracts as described above, the ratio of odd-chain fatty acids in the triglycerides produced by the cultured algae was significantly reduced, and therefore the cell extracts could not be used as a nitrogen source for the medium when efficiently producing the object of the present invention. Therefore, the present inventors found that the production of odd-chain fatty acids dramatically increased when Aurantiochytrium algae were cultured in an algae culture medium prepared by adding cell extracts treated with strong acid, compared to the case where cell extracts without such treatment were added. The method for producing triglycerides containing odd-chain fatty acids as a major component has already been reported (JP 2017-063633 A).

Furthermore, in one preferred embodiment of the invention, the basic medium for cultivating algae of the genus Aurantiochytrium is a medium with 2% or more glucose, 0.5-4% monosodium glutamate, 0.1-2% yeast extract, 1-3.3% sea salt, and 2-20% whey (animal or vegetable) supplemented with 0 to 50 mM of valine and 0 to 50 mM of sodium propionate. Animal or vegetable whey is preferably tofu whey (soybean whey). To this basic medium, add 2% or more of a culture of Aurantiochytrium that has been pre-cultured for 72 hours at 20-30° C. with 2% or more glucose, 0.5-4% monosodium glutamate, 0.1-2% yeast extract, 1-3.3% sea salt, and 2-20% whey (animal or vegetable). The Aurantiochytrium-added culture medium is aerated and gently agitated, and incubated for 48 to 200 hours at 20 to 30° C. and pH of 5.0 to 8.5 (use 1.0M NaOH solution for pH adjustment). After culturing, Aurantiochytrium cells that produced pentadecanoic acid triglyceride can be recovered by centrifugation (see the specification of JP Patent Application No. 2018-171739).

The pellets collected from the culture medium obtained by the above method by centrifugation or filtration are dried by freeze-drying or drying by heating. Alternatively, the medium in which the algal cells are suspended after cultivation may be used directly for the triglyceride extraction step. Extraction may be carried out multiple times using different organic solvents. As organic solvents, mixtures of polar and weakly polar solvents such as n-hexane/ethanol mixtures, chloroform/methanol mixtures, or ethanol/diethyl ether mixtures can be used. The obtained extracts are purified by methods known to those skilled in the art.

As a method for separating triglycerides, a fractionation method known to those skilled in the art is adopted. Separation and purification may be performed using various physicochemical properties such as the polarity of the triglyceride molecule to be fractionated, the solubility in a solvent, the melting point, the specific gravity, and the molecular weight, and column chromatography technology is preferably used. The conditions for the means of separating the triglycerides can be set by the normal condition study of the person skilled in the art, depending on the composition of the triglyceride mixture and the type of triglyceride to be fractionated.

The algae of the genus Aurantiochytrium can synthesize and accumulate both odd-chain fatty acid triglycerides and highly unsaturated fatty acid triglycerides intracellularly. Therefore, hexane or ethyl acetate is added to the obtained Aurantiochytrium cells to extract lipids, then hydrogen peroxide solution is added to this lipid solution (water is added if necessary), and unsaturated fatty acids are oxidized and decomposed by venting ozone at room temperature. After completion of the reaction, the oxide is removed with sodium hydrogen carbonate and sodium carbonate or an ion exchange resin to obtain pentadecanoic acid triglyceride that precipitates as the temperature decreases. The composition of the purified pentadecanoic acid triglyceride can be analyzed by gas chromatography, mass spectrometry and the like.

### (Collagen Production Promoter and Wrinkle Improver)

Collagen production promoter is a preparation that has the effect of increasing the production of collagen in human or animal cells. Administration of collagen production promoter to humans and other animals can prevent and/or ameliorate the decrease in the amount of collagen in the body due to decreased production or increased degradation of collagen. It can then be used to prevent and/or improve disorders (diseases) and age-related changes that are appeared due to a decrease in the amount of collagen in the body, such as osteoporosis, osteoarthritis, arteriosclerosis, cerebral hemorrhage, periodontal disease, and skin wrinkles.

The application of the collagen production promoter is not limited and can be set according to the purpose. For example, it can be used as a topical skin product or a beauty food or beverage. In addition, it can be applied to living organisms to improve skin conditions and promote wound healing. It can also be used to promote the formation of collagen-containing tissues and bone tissues such as artificial tissues and scaffold materials by adding it to culture media and culture solutions. When applied to a subject, the route of application is not particularly limited, and for example, transdermal, transmucosal, topical, intradermal, subcutaneous, intramuscular, ocular, intravitreal, subconjunctival, suprachoroidal, intraocular, oral, inhalation, intrabronchial, intrapulmonary, intravenous, intraarterial, intragastric, intraduodenal, intra-abdominal, intra-bladder, intra-vaginal, and the like.

The collagen that can be promoted for production is not limited to type I collagen, type II collagen, and other collagens, but can be used especially for the production of type I collagen, which is abundant in bones and skin. Type I collagen consists of two a1 chains and one a2 chain, and is known to be abundant in bones and skin as described above. It is known that promoting the production of type I collagen and maintaining the amount of type I collagen is effective in preventing and improving wrinkles and sagging. Furthermore, promoting the production of type I collagen is also effective in improving wound healing in the skin.

It is also known that in bone, 80% of the organic matter is composed of type I collagen, and calcium phosphate is deposited around the type I collagen fibers secreted by osteoblasts to form hydroxyapatite, which in turn forms bone. Therefore, by promoting the production of type I collagen, bone is regenerated, which can be applied to the treatment of osteoporosis.

The content of the active ingredients in the collagen production promoter and/or wrinkle improver of this embodiment is not particularly limited and can be determined as appropriate. From the viewpoint of demonstrating the collagen production-promoting effect and the wrinkle-improving effect, for example, 0.001% by mass or more is preferable, 0.01% by mass or more is more preferable, and 0.1% by mass or more is more preferable with respect to the total mass of the composition of the respective agents. The upper limit of the content in demonstrating each action is 100% by mass. However, as described below, when preparing topical skin care products, beauty foods and beverages, etc. by blending the collagen production promoter and/or the wrinkle improver, the content of the active ingredients should follow the following description of the respective products.

### (Topical Skin Care Product)

The topical skin care products of the present embodiment includes basic cosmetics such as skin toners, emulsions, creams, ointments, lotions, oils and packs; skin cleansing agents such as soaps, cleansing creams, cleansing lotions and facial cleansers; cosmetics for hair washing such as shampoos, rinses and treatments; hair cleanser, hair nourisher, hair growth agent such as hair creams, hair sprays, hair tonics, hair gels, hair lotions, hair oils, hair essences, hair waters, hair waxes and hair foams; makeup cosmetics such as foundations, white powders, face powders, lipsticks, cheek reds, eye shadows, eyeliners, mascaras, eyebrow inks and eyelashes; finishing cosmetics such as beauty nails; cosmetic compositions such as perfume, oral compositions such as toothpastes, and mouthwashes; medicated oral compositions such as topical medicinal preparations; ointments; haps; bath agents; medicated toothpastes; medicated oral compositions such as mouth fresheners; medicated cosmetics; inhibitors/deodorant of hair solvent odors such as hair dyes, hair growth agents, hair loss prevention agents, and hair removal agents; topical medical products, and topical pharmaceuticals such as hygiene products, hygiene cotton, and wet tissues.

These topical skin care products contain other ingredients customarily used in cosmetics, quasi-drugs and pharmaceuticals, such as powder ingredients, liquid fats, solid fats, waxes, hydrocarbons, higher fatty acids, higher alcohols, esters, silicones, anionic surfactants water-soluble polymers, thickeners, film agents, ultraviolet absorbers, metal ion sequestering agents, lower alcohols, polyhydric alcohols, sugars, amino acids, organic amines, polymer emulsions, pH adjusters, skin nutrients, vitamins, antioxidants, antioxidant auxiliaries, fragrances, water, and the like can be added as necessary and manufactured by conventional methods.

Other ingredients that can be used in topical skin care products include preservatives (ethyl paraben, butyl paraben, etc.), anti-inflammatory agents (e.g., glycyrrhizic acid derivatives, glycyrrhetinic acid derivatives, salicylic acid derivatives, hinokitiol, zinc oxide, allantoin, etc.), whitening agents (e.g., ascorbic acid and derivatives thereof, placenta extracts, saxifrage extracts, arbutin, etc.), various extracts (e.g., phellodendron bark, coptis rhizome, lithospermum root, peony root, swertia herb, birch, sage, loquat, carrot, aloe, hollyhock, iris, grape, iokinin, loofah, lily, saffron, cnidium rhizome, ginger, Hypericum erectum, Ononis spinosa, garlic, Capsicum annuum, citrus unshiu peel, Angelica acutiloba, seaweed, etc.), stimulants (e.g., royal jelly, photosensitizers, cholesterol derivatives, etc.), blood circulation stimulants (e.g., walenylamide nonylate, benzyl nicotinate ester, beta-butoxyethyl nicotinate ester, capsaicin, etc.), and anti-inflammatory agents (e.g., corticosteroids, corticosteroids, etc.). Butoxyethyl ester, Capsaicin, Zingerone, Cantharides tincture, Ictamol, Tannic acid, alpha-borneol, Tocopherol nicotinate, Inositol hexanicotinate, Cyclanderate, Cinnamalizine, Trazoline, Acetylcholine), anti-seborrheic agents (e.g., sulfur, thianthol, etc.), anti-inflammatory agents (e.g., tranexamic acid, thiotaurine, hypotaurine, etc.), and fungicides (e.g., triclosan, cetylpyridinium chloride, thymols, benzalkonium chloride, etc.). benzalkonium chloride, etc.), etc.

The target of administration of the topical skin care products of this embodiment is preferably warm-blooded vertebrate animals, and more preferably mammals. Mammals herein include, for example, humans, and non-human mammals such as monkeys, mice, rats, rabbits, dogs, cats, cattle, horses, and pigs. The topical skin care products of this embodiment is preferably applied to human subjects who desire to promote collagen production in the skin, improve wrinkles, add firmness or elasticity to the hair, or improve the texture of the hair.

The dosage of the active ingredient in the topical skin care composition of the present embodiment can be determined as appropriate depending on the condition of the individual, body weight, sex, age, activity of the material, route of administration or intake, administration or intake schedule, formulation form, or other factors. For example, based on the mass of the active ingredient, the dosage is preferably 0.001 mg or more, more preferably 0.01 mg or more, preferably 1000 mg or less, more preferably 100 mg or less, preferably 0.001 to 1000 mg, more preferably 0.01 to 100 mg, per day, per adult (60 kg body weight). In addition, the active ingredient may be ingested and administered once to several times a day, or at any periods and intervals.

The content of the active ingredient in the topical skin care composition of the present embodiment can be appropriately determined to achieve the above dosage. For example, in the total amount of the topical skin care composition of the present embodiment, the content of the active ingredient is preferably 0.001% by mass or more, more preferably 0.01% by mass or more, preferably 10% by mass or less, and 5% by mass. The range of 0.001 to 10% by mass is preferable, and 0.01 to 5% by mass is more preferable.

### (Beauty Food or Beverage)

The beauty food or beverage of the present embodiment contains pentadecanoic acid triglyceride represented by formula (I) above as an active ingredient and has a collagen production promoting action and a wrinkle improving action, and can prevent and/or improve skin aging through the respective actions, making it suitable for use in beauty food or beverage. Here, “beauty food or beverage” means food or beverage for the purpose of beautiful skin or preventing and improving skin aging.

The beauty food or beverage may be formulated in any drink or food that does not interfere with the above collagen production promoting and wrinkle improving effects, or it may be a dietary supplement in which the collagen production promoters and wrinkle improvers of the present invention are the main ingredients.

In the manufacture of beauty food or beverage containing pentadecanoic acid triglyceride represented by formula (I) above, for example, sugars such as dextrin and starch; proteins such as gelatin, soy protein, and corn protein; amino acids such as alanine, glutamine, and isoleucine; polysaccharides such as cellulose and gum arabic; oils and fats such as soybean oil and medium-chain fatty acid triglycerides can be added to formulate any dosage form.

The amount of pentadecanoic acid triglyceride represented by the above formula (I) in the beauty food or beverage of this embodiment should be adjusted so that the intake of pentadecanoic acid triglyceride per adult per day is about 1 to 1000 mg per day, considering the general intake of the food or beverage to be added.

Specific examples of the above-mentioned beauty food or beverage include, for example, beverages such as soft drinks, carbonated drinks, nutritional drinks, fruit drinks, and lactic acid drinks (including concentrates of these beverages and powders for adjustment); ice confections such as ice cream, ice sherbet, and shaved ice; noodles such as soba, udon, harusame, gyoza skin, siumai skin, chinese noodles, and instant noodles; confectionery such as sweet, candy, gum, chocolate, snacks, cookies, jelly, jam, cream, baked goods; processed marine and livestock products such as fish paste, ham, sausage; dairy products such as processed milk, fermented milk; fats and oils and processed foods thereof such as salad oils, tempura oils, margarines, mayonnaises, shortenings, whipped creams, dressings; seasonings such as sauces and bastes; health and nutritional supplements in various forms such as tablets and granules; others such as soups, stews, salads, prepared foods, and pickles.

The beauty food or beverage of the present embodiment may contain various food additives, such as antioxidants, flavors, various esters, organic acids, organic acid salts, inorganic acids, inorganic acid salts, inorganic salts, dyes, emulsifiers, preservatives, seasonings, sweeteners, acidifiers, fruit juice extracts, vegetable extracts, nectar extracts, pH adjusters, quality stabilizers, and the like, alone or in combination.

The concentration of pentadecanoic acid triglyceride in the beauty food or drink according to the present embodiment is about 0.00001 to 100% by mass (hereinafter referred to as “%”) as a solid content, and preferably 0.0005 to 50% to obtain usability and good effects.

Next, the following examples illustrate the present invention in more detail, but the present invention is not limited to these examples.

### EXAMPLES

### (Production Example 1) Production of Pentadecanoic Acid Triglyceride Using Aurantiochytrium

Aurantiochytrium, strain mh1959 (purchased from Professor Masahiro Hayashi, Faculty of Agriculture, Miyazaki University) was pre-cultured at 25° C. for 72 hours using a medium containing 3.6% glucose, 0.5% monosodium glutamate, 0.2% yeast extract, 1% seawater salt, and 10% whey. This was added to the following basal medium to make it 2% and gently agitated with air. 1 kg of basal medium was prepared by adding 50 mM valine and 25 mM sodium propionate to a medium containing 3.6% glucose, 0.5% monosodium glutamate, 0.2% yeast extract, 1% sea salt, and 10% whey. The culture medium was maintained at 25° C., pH 7.40-7.75 (1.0 M NaOH solution was used for pH adjustment) and incubated for 72-96 hours.

After incubation, the algae were centrifuged at 3000 rpm for 15 minutes and about 20 g of algae were collected. The lipids were extracted by adding hexane or ethyl acetate to 20 g of the obtained algae. Hydrogen peroxide solution was added to the extracted lipid solution (water was added as necessary), and ozone was vented at room temperature. After the reaction was completed, the oxides were removed using sodium bicarbonate, sodium carbonate, or ion exchange resin, and 2 g of pentadecanoic acid triglyceride was obtained, which precipitated as the temperature decreased.

### (Composition Analysis of Pentadecanoic Acid Triglyceride)

Methyl ester of fatty acid (FAME) was obtained by adding 0.50 mL of 14% BF₃-methanol and 0.25 mL of methyl acetate to the lipid containing pentadecanoic acid triglyceride obtained in Production Example 1 and heated at 70° C. for 30 min. Exactly 1.0 mL of n-hexane and 5 mL of saline were added to the reaction solution and mixed vigorously. The mixture was centrifuged at 2800 rpm for 10 min, and the n-hexane layer was used as the sample for gas chromatography.

The above samples were analyzed using a Shimadzu GC-2025 gas chromatograph system. The analysis conditions were as follows: Agilent J&W GC column DB-23 (30 m×0.25 mm) was used, 1 μL sample was injected and detected by FID (hydrogen flame ionization detector) with carrier gas (He, 14 psi). The molecular species of FAME was identified based on the retention time of the fatty acid methyl ester standard product (manufactured by GL Science Co., Ltd.). The fatty acid composition was determined from the area ratio. The obtained component ratios are mass ratios. The ratio of odd-chain fatty acids was determined by multiplying the total amount of fatty acids by the respective ratio (%) of odd-chain fatty acids (C13, C15, C17). The results obtained are shown in Table 1 below.

TABLE 1

| | Retention | | Ratio |
|---|---|---|---|
| Fatty acid | time (min) | Peak area | (mass %) |
| C11:0 | 7.939 | 2207 | 0.3 |
| C12:0 | 8.483 | 2735 | 0.3 |
| C13:0 | 9.065 | 39249 | 5.3 |
| C14:0 | 9.715 | 99919 | 13.4 |
| C15:0 | 10.461 | 422015 | 56.7 |
| C16:0 | 11.291 | 134108 | 18.0 |
| C17:0 | 12.23 | 44681 | 6.0 |

From the results shown in Table 1, the content of odd-chain fatty acids in the triglyceride obtained in Production Example 1 was 68.3% by mass. The fatty acids that make up the triglycerides were found to be mainly pentadecanoic acid residues (C15) and palmitic acid residues (C16).

### (Mass Spectrometry of Pentadecanoic Acid Triglyceride)

Lipids containing pentadecanoic acid triglyceride obtained in Production Example 1 were analyzed by mass spectrometry using a Thermo Fischer Orbitrap mass spectrometer Exactive Plus (AMAR DART ion source). The results are shown in Table 2 below.

TABLE 2

| Total carbon number of | | | | Relative | | |
|---|---|---|---|---|---|---|
| acyl groups | tR | Area(%) | m/z | Intensity | fragment(%) | Assignment |
| | 33.2 | 18.8 | 768.7079 | 86.62 | 0 | M + NH4 |
| | | | 495.441 | 14.25 | 8.626952 | M − C16 |
| C14•C15•C15 | | | 509.4569 | 100 | 60.540017 | M − C15 |
| | | | 523.4725 | 44.26 | 26.795012 | M − C14 |
| | | | 537.4882 | 6.67 | 4.038019 | M − C13 |
| | | | 551.5039 | 0 | 0 | M − C12 |
| | 38.6 | 30.6 | 782.7241 | 91.24 | 0 | M + NH4 |
| | | | 509.4567 | 19.87 | 14.877209 | M − C16 |
| C15•C15•C15 | | | 523.4726 | 100 | 74.872716 | M − C15 |
| | | | 537.4877 | 13.69 | 10.250075 | M − C14 |
| | 46 | 22.3 | 796.7401 | 100 | 0 | M + NH4 |
| | | | 509.4566 | 6.21 | 5.607730 | M − C17 |
| C16•C15•C15 | | | 523.4725 | 41.7 | 37.655770 | M − C16 |
| | | | 537.4882 | 58.44 | 52.772259 | M − C15 |
| | | | 551.5033 | 4.39 | 3.964241 | M − C14 |
| | 55.8 | 11.7 | 810.7554 | 100 | 0 | M + NH4 |
| C17•C15•C15 | | | 523.4721 | 35.42 | 19.954930 | M − C17 |
| C16•C16•C15 | | | 537.4878 | 65.54 | 36.923944 | M − C16 |
| | | | 551.5035 | 71.82 | 40.461972 | M − C15 |
| | | | 565.5186 | 4.72 | 2.659155 | M − C14 |

The results in Table 2 show that the pentadecanoic acid triglyceride obtained in Production Example 1 contains mainly triglycerides formed only with pentadecanoic acid residue (C15) and triglycerides containing two units of pentadecanoic acid residue (C15) and one unit of palmitic acid residue (C16), based on the fragment composition of the major mass spectral peaks (peaks 4 and 5).

### (Verification of the Effect of Promoting the Production of Type I Collagen in Human Dermal Fibroblasts)

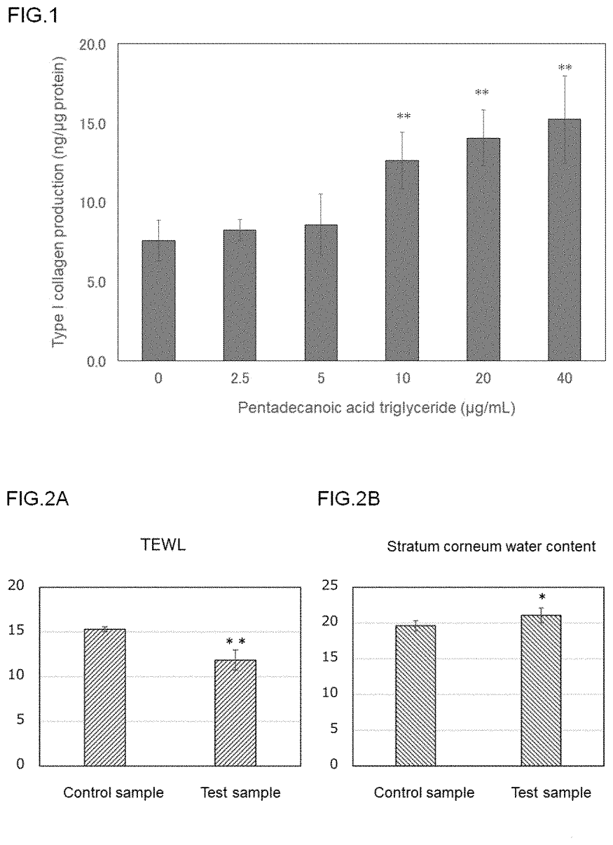

Human dermal fibroblasts were seeded in 96-well plates containing Dulbecco's Modified Eagle Medium (DMEM) containing 0.5% fetal bovine serum (FBS) at a density of 2.0×10⁴ cells/well and incubated for 24 hours. The culture medium was changed to DMEM containing 0.5% FBS supplemented with pentadecanoic acid triglyceride (PdATG) obtained by Production Example 1 at various concentrations from 0 to 40 μg/mL, and incubated for another 24 hours. The culture supernatant was collected, and the amount of type I collagen contained was quantified by ELISA. The total protein content of the cells was measured by the BCA method, and the results of the amount of type I collagen produced per unit cellular protein are shown in Table 3 and FIG. 1.

TABLE 3

| Sample | | | |
|---|---|---|---|
| concentration | Average value | Standard | |
| (μg/mL) | (ng/μg protein) | deviation | p-value |
| 0 | 7.59 | 1.28 | — |
| 2.5 | 8.26 | 0.66 | 0.384 |
| 5 | 8.59 | 1.93 | 0.418 |
| 10 | 12.65 | 1.77 | 0.007 |
| 20 | 14.07 | 1.75 | 0.002 |
| 40 | 15.24 | 2.75 | 0.002 |

In Table 3 and FIG. 1, the measured values of type I collagen are the average and standard deviation values of the four samples at each concentration, and any significant difference at p<0.01 in the Student's t-test compared to the mean value when the sample concentration was 0 μg/mL is indicated by **. From the results in Table 3 and FIG. 1, it was confirmed that the amount of type I collagen produced by human fibroblasts increased according to the concentration of pentadecanoic acid triglyceride (PdATG) added to the culture medium.

### (Verification of the Effect of Oral Administration on the Maintenance of Skin Moisturizing Properties)

We examined the effect of oral administration of pentadecanoic acid triglyceride obtained in Production Example 1 on the maintenance of moisture retention in hairless mice. The test was conducted by measuring and evaluating the trans epidermal water loss (TEWL) and stratum corneum water content using 5-week-old hairless mice (strain Hos: HR-1) supplied by SLC Japan. The test samples were prepared by dissolving the test substance containing pentadecanoic acid triglyceride in corn oil (30 w/v %). One group of five animals was orally administered 0.2 mL of the test sample or the control sample once a day for 14 consecutive days using a sonde.

TEWL and stratum corneum water content were measured using a Tewameter TM300 and a Corneometer CM825 (both manufactured by Courage+Khazaka electronic GmbH) in that order. The measurements were taken in the left ventral part of the midline in mice under 20% isoflurane inhalation. The measurement settings were as follows: for TEWL, the acquisition interval was 1 second, the number of moving averages was 5, the end condition was one measurement with a standard deviation of 0.3 or less, and for stratum corneum water content, the average value of 10 measurements was obtained. The significance test was set at the significance level; **: p<0.01, *: p<0.05. The results are shown in FIG. 2.

FIG. 2A shows the results of trans epidermal water loss (TEWL) measurements, where the vertical axis represents the evapotranspiration rate (g/h/m²). The average value±standard deviation of five measurements on the abdomen of hairless mice treated with the control sample was 15.3±0.27. In contrast, when the test sample was administered, the average value was 11.8±1.14, indicating a significant decrease in skin water evaporation. FIG. 2B shows the results of the measurement of stratum corneum water content in the abdomen of hairless mice. The average value±standard deviation of 10 times for the mice treated with the control sample was 19.62±0.72, whereas the average value±standard deviation of 10 times for the mice treated with the test sample was 21.08±1.04, indicating a significant increase in stratum corneum water content.

As a result of examining the effect of oral administration of test substances including pentadecanoic acid triglyceride on the maintenance of moisturizing properties in hairless mice, trans epidermal water loss (TEWL) and stratum corneum water content, which are indices to evaluate moisturizing properties, were significantly improved compared to the control. The results showed that oral administration of pentadecanoic acid triglyceride enhanced the moisturizing property of the skin.

### (Topical Skin Care Product)

Using the pentadecanoic acid triglyceride obtained in Production Example 1, the preparation was made in the following blending ratio to prepare a topical skin cream as one embodiment of the topical skin care product.

Stearate 2% by mass

Glycerin stearate 1.2% by mass

Cetyl ethylhexate 2.4% by mass

Setostearis alcohol 1.8% by mass

Squalane 0.6% by mass

Pentadecanoic acid triglyceride 0.01% by mass

Trimethylsiloxyphenyl dimethicone 2.9% by mass,

PEG-40 stearate, 1.2% by mass,

methylparaben, 0.2% by mass,

10% Na hydroxide, 2% by mass,

carboxyvinyl polymer, 0.2% by mass,

xanthan gum, 0.1% by mass,

glycerin, 3% by mass,

BG, 3% by mass,

Purified water 79.39% by mass

### (Wrinkle Improver)

Using the pentadecanoic acid triglyceride obtained in Production Example 1, the wrinkle improving cream was prepared as one embodiment of the wrinkle improver by blending in the following blending ratio.

Glyceryl stearate 1.0% by mass

Cetesu-20 1.0% by mass

Solves tetraoleate-40 1.0% by mass

Cetearyl alcohol 6.0% by mass

Olive squalane 8.0% by mass

Pentadecanoic acid triglyceride 0.1% by mass

Triethylexanoin 8.0% by mass

Xanthan gum 15.0% by mass

Pedilauroyl glutamate lysine Na 1.0% by mass

BG 3% by mass

Purified water 55.9% by mass

### (Beauty Food or Beverage)

Pentadecanoic acid triglyceride obtained in Production Example 1 was used and blended in the following proportions to prepare beverage water for beauty as one embodiment of the beauty food or beverage. The following is the amount of the component in 100 mg of beverage water.

Pentadecanoic acid triglyceride 10 mg,

Coenzyme Q10 10 mg,

Vitamin B2 5 mg,

vitamin B6 5 mg,

nicotinamide 10 mg,

multidextrin 60 mg

The above-exemplified examples of compositions of topical skin products, wrinkle improvers, and beauty foods or beverages are provided for the purpose of facilitating understanding of the topical skin products, wrinkle improvers, and beauty foods or beverages in accordance with the mode of implementation of the present invention, and the present invention is in no way limited to these compositions. We believe that a person skilled in the art can easily understand that these exemplified compositions can be changed or modified in various ways based on the above-mentioned description within the scope of the present invention, including the scope of the claims.

1. A collagen production promoter comprising, as an active ingredient, a triglyceride represented by the following formula (I): 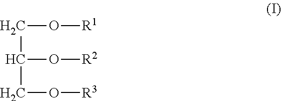
wherein R¹, R² and R³ each denotes a saturated fatty acid residue, at least one of which is a pentadecanoic acid residue.

2. The collagen production promoter according to claim 1, wherein R¹ and R², or R¹ and R³ are pentadecanoic acid residues.

3. The collagen production promoter according to claim 1, wherein any one of R¹, R² and R³ is a myristic acid residue (C14), a palmitic acid residue (C16) or a margaric acid residue (C17).

4. The collagen production promoter according to claim 1, comprising a triglyceride represented by formula (I) of claim 1 wherein R¹, R² and R³ all denote pentadecanoic acid residues, and a triglyceride represented by formula (I) of claim 1 wherein any two of R¹, R² and R³ are pentadecanoic acid residues and another is a myristic acid residue or a palmitic acid residue.

5. A wrinkle improver comprising the triglyceride represented by formula (I) of claim 1.

6. A topical skin care composition comprising the triglyceride represented by formula (I) of claim 1.

7. The topical skin care composition according to claim 6 for use in promoting collagen production and/or improving wrinkle of skin.

8. A beauty food or beverage comprising, as an active ingredient, the triglyceride represented by formula (I) of claim 1.

9. The beauty food or beverage according to claim 8 for use in promoting collagen production and/or improving wrinkle of skin.

10. A triglyceride derived from algae of the genus Aurantiochytrium, represented by the following formula (I): 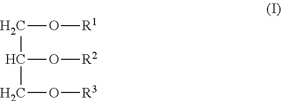
wherein R¹, R² and R³ each denotes a saturated fatty acid residue, at least one of which is a pentadecanoic acid residue.

11. The triglyceride according to claim 10, wherein any two of R¹, R² and R³ are pentadecanoic acid residues and another is a myristic acid residue or a palmitic acid residue.

12. A method for improving wrinkle of skin in a subject in need thereof, comprising administering to the subject a composition containing an effective amount of triglyceride represented by the following formula (I): 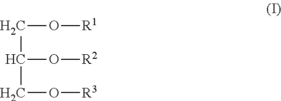
wherein R¹, R² and R³ each denotes a saturated fatty acid residue, at least one of which is a pentadecanoic acid residue.

13. The method for improving wrinkle of skin in a subject according to claim 12, wherein the composition is a topical skin care composition.

14. The method for improving wrinkle of skin in a subject according to claim 12, wherein the composition is a food or beverage composition.
